(19) United States
(12) Patent Application Publication
(10) Pub. No.: US 2004/0115749 A1
(43) Pub. Date: Jun. 17, 2004

(54) Cardiovascular disease and thrombotic risk diagnostic tests

(76) Inventor: Michael R. Buchanan, Hamilton (CA)

Correspondence Address: JACOBSON HOLMAN PLLC, 400 SEVENTH STREET N.W., SUITE 600, WASHINGTON, DC, 20004, US

(21) Appl. No.: 10/474,037

(22) PCT Filed: Apr. 12, 2002
(86) PCT No.: PCT/CA02/00511

(30) Foreign Application Priority Data
Apr. 12, 2001 (CA) 2344007

(51) Int. Cl. G01N 33/53; G01N 33/537; G01N 33/543

(52) U.S. Cl. 435/7.92

(57) ABSTRACT

Measuring plasma 13-HODE is a useful maker to assess i) the severity or burden of coronary artery disease and ii) the risk of thrombotic events in patients undergoing medical and/or surgical procedures for revascularization.

### FIELD OF INVENTION

In general, this invention relates diagnostic kits for assessing thrombotic risk and burden of cardiovascular disease.

### BACKGROUND

Cardiovascular disease (CVD) is a leading cause of morbidity and death in Western societies. Each year there are >1,000,000 percutaneous transluminal coronary angioplasty (PTCA) and surgically invasive procedures, e.g. coronary artery bypass grafting (CABG) in N. America alone, performed in cardiovascular disease patents to improve (cardio)vascular blood flow. Early assessment of cardiovascular risk factors before the onset of CVD or the assessment of burden of disease would enable practitioners to develop early intervening strategies to prevent or diminish the progression of the disease. Likewise, early identification of those individuals who are at thrombotic risk would enable practitioners to develop early-treatment programs for reducing the likelihood of a thrombotic event. Clearly, there is an urgent need to generate diagnostic kits for assessing thrombotic risk and/or burden of cardiovascular disease.

### SUMMARY

This invention is direct to a diagnostic kit for determining the concentration of 13(HODE) in a biological sample of a subject. In one embodiment 13(HODE) is 13(S)-HODE and in another embodiment 13(HODE) is 13(R)-HODE. In one embodiment the diagnostic kit is useful for assessing the risk or burden of-the subject to cardiovascular disease. In another embodiment the diagnostic kit is useful for assessing the thrombotic risk or burden of disease of the subject In one embodiment the kit comprises: (a) enzyme-conjugated 13(HODE), (b) enzyme-conjugated 13(HODE) substrate, and (c) anti-13HODE specific antibody. In one embodiment the enzyme-conjugated 13(HODE) is 13(HODE)-horseradish peroxidase, and in another embodiment the enzyme-conjugated 13(HODE) is 13(HODE) alkaline phosphatase.

In another embodiment the enzyme-conjugated 13(HODE) substrate is capable of undergoing a colorimetric change and in another embodiment the enzyme-conjugated 13(HODE) substrate is capable of emitting a fluorescent signal.

In yet another embodiment the kit comprises: (a) fluorochrome conjugated 13(HODE) and (b) anti-13-HODE specific antibody.

In one embodiment the kit comprises: (a) radioisotope conjugated 13(HODE) and (b) anti-13(HODE) specific antibody.

The biological sample may be from tissue fluid, blood, blood plasma, blood serum or urine.

In a preferred embodiment the kit further comprises a standard wherein the standard is a known quantity of 13(HODE).

The invention is also directed a diagnostic method for assessing the risk or burden of cardiovascular disease in a subject that may be performed with the above-described kit.

The invention is further directed a diagnostic method for assessing thrombotic risk or burden in a subject that may be performed with the above-described kit.

In one embodiment the diagnostic method comprises the steps of (a) extracting 13(HODE) from a biological sample, (b) incubating extracted 13(HODE) with enzyme-conjugated 13(HODE) and anti-13(HODE) specific antibody bound to an insoluble support, (c) incubating the mixture resultant from step (b) with enzyme-conjugated 13(HODE) substrate, (d) determining by optical density measurements the concentration of 13(HODE) that was present in the biological sample based on the color signal produced and (e) comparing the concentration of 13(HODE) that was present in the biological sample to the concentration of 13(HODE) in biological samples form healthy subjects.

In another embodiment the method comprises the steps of (a) extracting 13(HODE) from a biological sample, (b) incubating extracted (13)HODE with enzyme-conjugated 13(HODE) and anti-13(HODE) specific antibody bound to an insoluble support, (c) incubating the mixture resultant from step (b) with enzyme-conjugated 13(HODE) substrate, (d) determining by fluorescence the concentration of 13HODE that was present in the biological sample based on the fluorescent signal produced, and (e) comparing the concentration of 13(HODE) that was present in the biological sample to the concentration of 13(HODE) in biological samples from healthy subjects.

In another embodiment the method comprises the steps of (a) extracting 13(HODE) from a biological sample, (b) incubating extracted 13(HODE) with fluorochrome conjugated 13(HODE) and anti-13(HODE) specific antibody bound to an insoluble support, (c) determining by fluorescence the concentration of 13(HODE) that was present in the biological sample based on the fluorescent signal produced, and (d) comparing the concentration of 13(HODE) that was present in the biological sample to the concentration of 13(HODE) in biological samples from healthy subjects.

In yet another embodiment the method comprises the steps of (a) extracting 13(HODE) from a biological sample, (b) incubating extracted 13(HODE) with radioisotope conjugated 13(HODE) and anti-13(HODE) specific antibody bound to an insoluble support, (c) determining by radiation spectrometry the concentration of 13(HODE) that was present in the biological sample based on the radiation produced, and (d) comparing the concentration of 13(HODE) that was present in the biological sample to the concentration of 13(HODE) in biological samples from healthy subjects.

### BRIEF DESCRIPTION OF THE DRAWINGS

Preferred embodiments of the invention will be described in relation to the drawings in which:

FIG. 1. 13(S)-HODE levels in saphenous veins obtained from patients undergoing CABG relative to patient age (years).

FIG. 2. Plasma, 13(S)-HODE in ‘healthy’ volunteers, in patients who underwent recent CABG and in patients who underwent CABG and were followed by 2 years.

FIG. 3. Plasma 13(S)-HODE (ng/ml) in non-smokers & smoker CVD patients who suffered or not a vascular event in the 2-year follow up after CABG. * p<0.01

FIG. 4. Receiver Operator Characteristics (ROC) Discriminatory Analysis

### DETAILED DESCRIPTION

The term “13-HODE” represents 13-hydroxyoctadeca-9Z, 11E-dienoic acid (13-HODE).

The term “antibody” as used herein refers to polyclonal antibodies or monoclonal antibodies.

The terms “enzyme-conjugated 13(S)-HODE” and “enzyme-conjugated 13(R)-HODE” as used herein refers to 13(S)HODE or 13(R)-HODE conjugated with an enzyme such as alkaline phosphatase, peroxidase, or the like, which is capable of reaction with an enzyme conjugated 13(S)-HODE or 13(R)-HODE substrate as defined below.

The terms “enzyme-conjugated 13(S)-HODE substrate” and “enzyme-conjugated 13(R)-HODE substrate” as used herein refers to a substrate for indicating, by colour change or change in fluorescence, the presence of enzyme-conjugated 13(S)-HODE or enzyme-conjugated 13(R)-HODE bound to anti-13(S)-HODE or anti-13(R)-HODE specific antibody, for example, p-nitrophenol phosphate is one such enzyme conjugated 13(S)-HODE or 13(R)-HODE substrate for the enzyme alkaline phosphatase.

The terms “fluorochrome conjugated 13(S)-HODE” and “fluorochrome conjugate 13(R)-HODE” as used herein refers to 13(S)-HODE or 13(R)-HODE conjugated with a fluorochrome such as phycoerythrin, which is capable of indicating, by fluorescence, the presence of fluorochrome conjugated 13(SYHODE or fluorochrome conjugate 13(R)-HODE bound to anti-13(S)-HODE or to anti-13(R)-HODE specific antibody.

The terms “radioisotope conjugated 13(S)-HODE” and “radioisotope conjugated 13(R)-HODE” as used herein refers to 13(S)-HODE or 13(R)-HODE conjugated to a radioisotope, such as H³, which is capable of indicating, by producing radiation, the presence of radioisotope conjugated 13(S)-HODE or radioisotope conjugated 13(R)-HODE bound to anti-13(S)-HODE or anti-13(R)-HODE specific antibody.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

The observation that the plasma 13(S)-HODE levels correlated inversely with the vessel wall levels is surprising. There are a number of possible explanations for such results.

First, it is possible that the 13(S)-HODE substrate, linoleic acid not only is not metabolized effectively by the vessel wall in cardiovascular diseased patients, but also is not incorporated into the vessel wall. Normally, linoleic acid is selectively incorporated in vessel wall cells rather than circulating blood cells. Consequently, the linoleic acid free in the plasma may be metabolized by blood cell lipoxygenases, thereby generating high plasma 13(S)-HODE levels.

Alternatively, linoleic acid may, in fact, be incorporated into the diseased vessel wall, but because of a defective lipoxygenase pathway, it may accumulate in vessel wall or inflammatory cells within the vessel wall; e.g. macrophages, and be metabolized non-enzymatically into 13-HODE-specifically 13(R)-HODE. This latter possibility seems more likely since other investigators have reported that racemic 13-HODE accumulates in thrombogenic plaques in patients with cardiovascular disease. The ELISA kit for 13(S)-HODE has 10% cross-reactivity with 13(R)-HODE. It is possible that the “13-HODE” detected in our assay is, in part, 13(R)-HODE.

Measuring plasma 13-HODE (13(R)-HODE±13(S)-HODE) is a useful tool for the assessment of severity of cardiovascular disease and the risk of possible (cardio)vascular thrombogenic events and are supported by the observations that i) plasma 13-HODE levels are higher in cardiovascular disease patients as compared with healthy volunteers in the same age range; ii) plasma 13-HODE levels increase with age in cardiovascular disease patients-the risk of (cardio)vascular thrombogenic events also increases with age; iii) plasma 13-HODE levels are higher in cardiovascular disease patients who suffer a (cardio)vascular thrombogenic event; and iv) plasma 13-HODE levels are highest in cardiovascular disease patients who also are smokers.

Previously, we reported that the vessel wall (VW) monohydroxide, 13-hydroxyoctadecadienoic acid (13-HODE) decreases with age, thereby increasing VW thrombogenicity in patients undergoing non-urgent coronary artery bypass grafting (CABG). This suggests that decreased VW 13-HODE increases the risk of thrombotic events in patients with coronary artery disease (CAD), and that low VW 13-HODE levels may be indicative of CAD severity. However, it is not possible to obtain VW segments from most CAD patients. Thus, we examined the relationship between VW and plasma 13-HODE levels with age in 47 male and 14 female patients undergoing non-urgent CABG and compared to the plasma 13-HODE levels in 22 healthy volunteers of the same age (35 to 75 years). We also compared the 13-HODE levels in plasma prepared at the time of surgery in 36 other CABG patients, 24 of who suffered a thrombotic event within 2 years of surgery.

Consistent with previous data, VW 13-HODE decreased with age; i.e., <50 years—243±100 ng/cm², mean±SD; 51-60 years—129±23 ng/cm²; and 61-75 years—108±39 ng/cm², p<0.05. Similar results were seen in male and female patients. In contrast, their plasma 13-HODE levels increased with age and were >10-fold higher than the levels seen in healthy volunteers, 3120 to >6000 ng/ml versus 214 to 670 ng/ml, respectively, p<0.005. Moreover, the plasma 13-HODE levels in the 24 patients who subsequently suffered a thrombotic event within 2 years of CABG were higher than in patients who did not suffer an event; 4500 to >6000 versus 4100 to 5308 ng/ml, p<0.01. The plasma 13-HODE levels were highest in those patients who were smokers; >6000 ng/ml, p<0.01.

These data demonstrate that measuring plasma 13-HODE is a useful marker to assess i) the severity or burden of coronary artery disease; and ii) the risk of thrombotic events in patients undergoing medical and/or surgical procedure for revascularization.

In addition, these data demonstrate that measuring plasma 13-HODE is a useful tool to assess cardiovascular disease severity and subsequent thrombotic risk.

Individuals who may be at thrombotic risk, include but are not limited to, individuals with cardiovascular disease, sickle cell disease, cancer, lupus, essential thrombocytopenia, hypocholesteranemia, diabetes, hypertension and hyperlipidemia, individuals with (anti)coagulant deficiencies or genetic anomalies such as seen with Factor V Leiden, protein C, protein S, antithrombin III, heparin II, prothrombin 20210GtoA variant or any other disease having a co-morbidity characteristic leading to a thrombophilic event.

Kits suitable for diagnosis and containing the appropriate labeled reagents are constructed by packaging the appropriate materials, including the conjugated 13(S,R)-HODE and the anti-13(S,R)-HODE specific antibody in suitable containers, along with the remaining reagents and materials required for the conduct of the assay, as well as a suitable set of assay instructions. Kits containing enzyme conjugated 13(S,R)-HODE will also contain the appropriate enzyme conjugated 13(S,R)-HODE substrate.

Any immunological test format is contemplated, such as enzyme linked immunosorbent assay (ELISA), Western blot, sandwich assay, a radioimmunoassay test, direct or indirect fluorescent antibody test (see: Current Protocols in Molecular Biology”, Wiley Interscience (1991)) and the like, which are well known to those skilled in the art.

The insoluble support to which the anti-13(S,R)-HODE specific antibody is bound may take various designs and shapes, such as, a sphere, plate, dish, small rod, cell, small bottle, small tube, fiber, network or the like.

Specific examples to be used include a microtiter plate made of transparent plastic material such as polyvinyl chloride or polystyrene, small sphere, tube, rod or the like made of polystyrene and polystyrene latex.

Another optional part of the kit is a set of instructions for use. The instructions describe the reagents included in the kit, procedures for reconstitution of reagents, stability and storage information, and information regarding the sucrose assay, including sample preparation, instrument settings, and calculations. Preferably the instructions are included in the form of a printed pamphlet.

### EXAMPLE 1

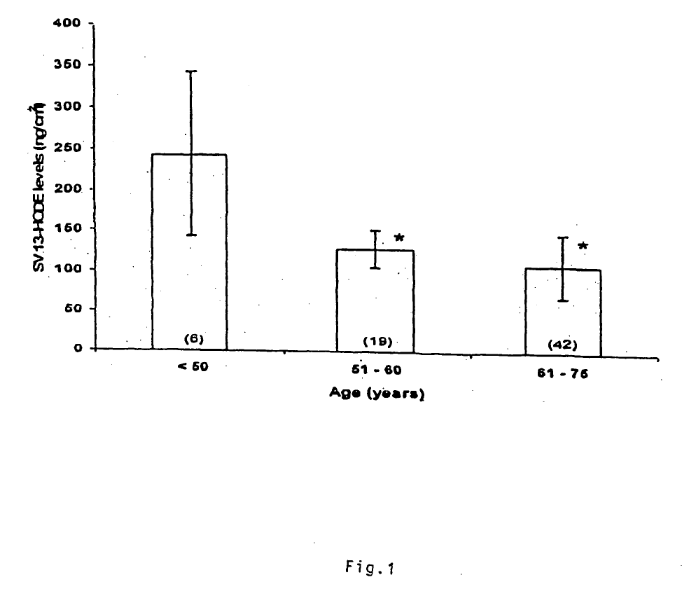

The endogenous 13(S)-HODE levels decreased with age in internal mammary arteries and in saphenous veins (Table 1; FIG. 1). The r values were 0.952, p<0.01 and 0.897, p<0.025, respectively. These decreases were seen in both male and female patients (data not shown).

### EXAMPLE 2

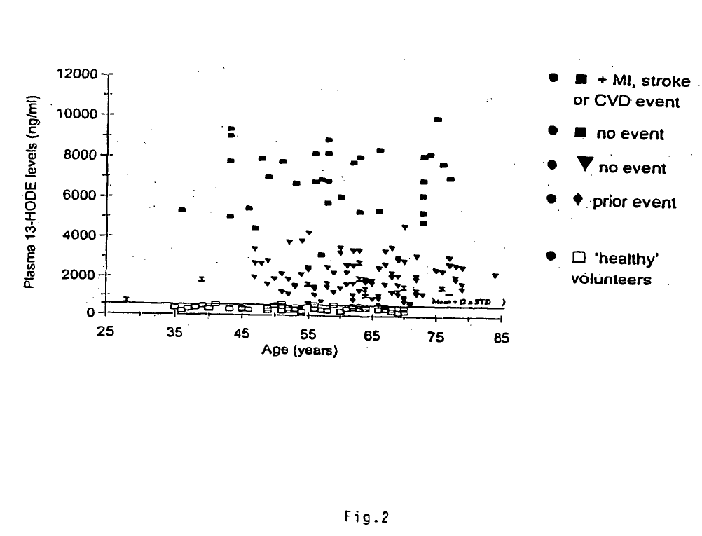

In contrast, the plasma 13-HODE levels increased dramatically. Thus, the plasma 13-HODE levels in patients <50 years of age was 1,811±326, mean±sem, and increased with age in both males and females (Table 2; FIG. 2). Thus, there was an inverse relationship between vessel wall 13(S)-HODE levels and plasma 13-HODE level, r=0.991, p<0.025.

The plasma levels in these patients were significantly higher than the plasma levels seen in healthy volunteers at similar ages (Table 3; FIG. 2).

### EXAMPLE 3

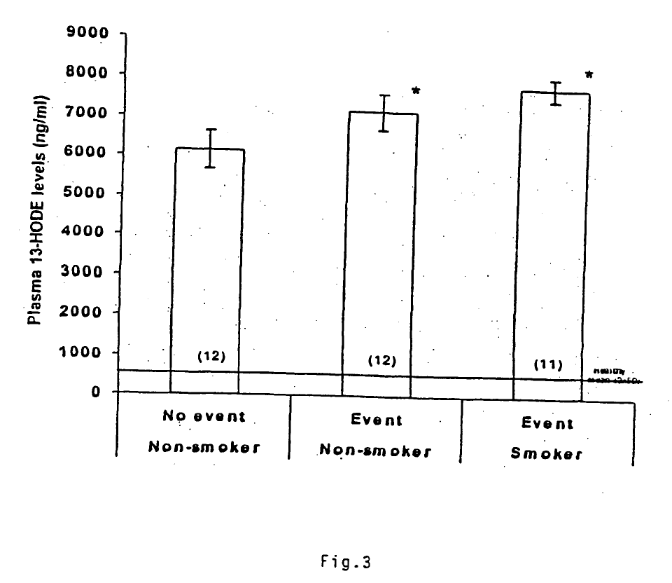

Consequently, we measured the plasma 13-HODE levels in 24 BRAT study patients who had suffered a (cardio)vascular thrombotic event in the 2 years following CABG and in 12 BRAT study patients who had not suffered a thrombotic event in the 2 years following surgery. The data are shown in Table 4 and FIG. 3.

### EXAMPLE 4

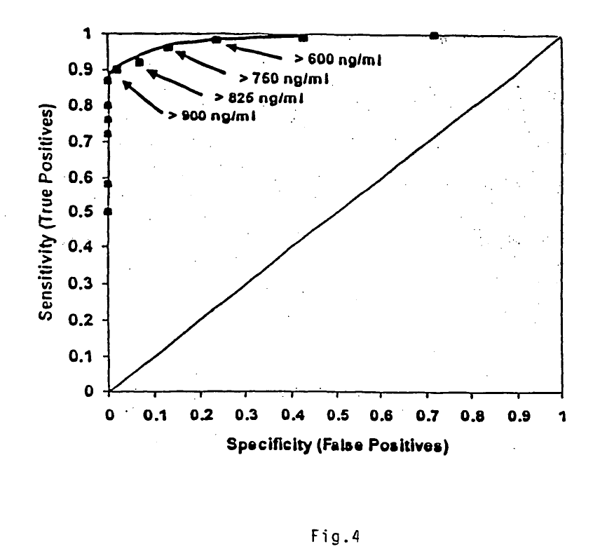

A Receiver Operator Characteristics (ROC) discriminatory analysis⁶ was performed on the data shown in FIG. 2 to determine the sensitivity (true positives) and selectivity (false positives) with respect to the identification of individuals with or without CVD. As shown in FIG. 4, measuring plasma 13-HODE as a predictor of CVD is highly sensitive and selective. For examples, for any individual who has a plasma level of ≧900 ng/ml, there is a 92% probability with a <2% error and with 93% confidence, that the individual has CVD.

TABLE 1
Internal Mammary Artery (IMA) and Saphenous Vein
(SV) 13(S)-HODE levels with Age in Vessels Obtained
from CABG Patients at the Time of Surgery.

| | <50 years | 51-60 years | 61-70 years | >70 years |
|---|---|---|---|---|
| Internal Mammary* | 351 ± 112 | 299 ± 33 | 239 ± 31 | 139 ± 14 |
| Saphenous Vein** | 253 ± 143 | 129 ± 9 | 112 ± 10 | 108 ± 9 |

*Data are expressed as ng/cm², means ± sem.r = 0.952, p < 0.01;
**r = 0.897, p < 0.025

TABLE 2
Plasma 13-HODE levels with Age in CABG Patients.
Plasma Samples Were Obtained at the Time of Surgery.

| | <50 years | 51-60 years | 61-70 years | >70 years |
|---|---|---|---|---|
| Males* | 1775 ± 205 | 1739 ± 164 | 2933 ± 732 | 3120 ± 1394 |
| Females** | 1532 ± 357 | 1616 ± 290 | 1953 ± 401 | ND |

*Data are expressed as ng/ml, means ± sem. r = 0.918, p < 0.025;
**r = 0845, p < 0.05. ND = no data

TABLE 3
Plasma 13-HODE levels with Age in Healthy Volunteers.

| | <50 years | 51-60 years | 61-70 years | >70 years |
|---|---|---|---|---|
| Males* | 441 ± 60 | 553 ± 100 | 532 ± 50 | ND |
| Females** | 419 ± 25 | 417 ± 25 | 523 ± 60 | ND |

*Data are expressed as ng/ml, means ± sem.,
ND = no data

TABLE 4
Plasma Levels in BRAT study Patient Who Did and
Did Not Suffer a (Cardio) vascular Thrombotic Event.

| | Plasma 13-HODE (ng/ml) |
|---|---|
| Patients with No Events | 4,100-5308 |
| Non-Smokers with Events | 4,500->6,000 |
| Smokers with Events | >6000 |

*Data are expressed the range, ng/ml.

Subjects

Blood samples obtained from 60 healthy Canadian Blood Services blood donors (both male and female, with ages ranging from 35 to 70 years) who had no history of cardiovascular disease (CVD), were used as the “healthy” control population.

Blood and vessel wall samples were collected during the CABG procedure from 139 male and CVD female patients undergoing non-urgent CABG. The vessel wall segments consisted of pieces of saphenous vein and/or internal mammary artery which were isolated during but not used in the surgical procedure.

Blood samples also were obtained from 24 patients (17 males, 7 females) who participated in the BRAT study and had suffered a (cardio)vascular event within two years of surgery, and from 12 patients (10 males and 2 females who participated in the BRAT study but did not suffered a (cardio)vascular event. The former were included to determine if plasma 13-HODE levels differed significantly in those patients as compared to patients who did not suffer an event post CABG. The latter were included as the appropriate controls for the former and as a comparison for the samples collected from patients during CABG in whom the two-year follow-up is incomplete and the samples collected from the ‘healthy’ control population.

The study protocol was approved by the individual Ethics committees of both health institutions, and each participant only was included following his/her being advised of the purpose nature of the study and the possible risks and benefits, and then signing and “Informed Consent” form.

Materials & Methods

Blood Samples: Each blood sample was collected into sodium citrate (3.2% final concentration). The hematocrit was measured on each sample, and then the sample was centrifuged at 1800 g to prepare cell-free plasma. A 500 μl aliquot of each plasma was transferred to an acid washed, silanized vial containing 1 ml of HPLC grade methanol. The vial was sealed under nitrogen and stored at −80° C. until assayed.

Vessel Wall Samples: Each vessel wall sample was stripped clean of its adventitia, and then rinsed in isotonic Ringers lactate to wash off any blood. The vessel wall segment was then slit open longitudinally and laid flat, endothelial side down on a piece of clear acetate. The vessel wall segment area was traced onto the acetate, using a permanent marker pen. Later, the vessel wall area would be calculated using a computer-assisted Image Analyser. The vessel wall segment was then transferred into an acid washed, silanized vial containing 1 ml of HPLC grade methanol. Each vial was sealed under nitrogen and stored at −80° C. until assayed.

13-HODE Assay: Each plasma and vessel wall sample was extracted as previously described, using a commercially available 13(S)-HODE ELISA kit. The 13-HODE concentration (expressed as ng/ml) was corrected for any dilution effect where relevant, by comparing the hematocrit of the blood sample with the pre surgery hematocrit of each individual. The 13(S)-HODE concentration of the vessel wall was expressed as ng/cm², based upon the assumption that a cm² of saphenous vein weighed 13±1 mg/cm² (mean±sem) and each internal mammary artery weighed 13±1 mg/cm² (mean±sem). The weight of each segment was determined to confirm the latter assumption.

References

1. Buchanan M R, Schwartz L, Bourassa M G, Brister S J, Peniston C M on behalf of the BRAT Investigators. Results of the BRAT study—A pilot study investigating the possible significance of ASA nonresponsiveness on the benefits and risks of ASA on thrombosis in patients undergoing coronary artery bypass surgery. Can J Cardiol 16(11): 1385 - 1390, 2000.

2. Buchanan M R, Brister S J. Anticoagulant and antithrombin effects of Intimatan, a heparin cofactor II agonist. Thromb Res 99: 603-612, 2000.

3. Oxford Biomedical Research. 13(S)-Hydroxyoctadecadienoic Immunoassay Kit, Product # EA 81, 2000.

4. Bertomeu M -C, Crozier G L, Haas T A, Fleith M, Buchanan M R. Selective effects of dietary fats on vascular 13-HODE, synthesis and platelet/vessel wall interaction. Thromb Res 59: 819-830,1990.

5. Breugnot C, Privat S, Guillonneau C et al. S12340: a potent inhibitor of the oxidative modification of low-density lipoprotein in vitro and in vivo in WHHL rabbits. J Pharmacol Exp Ther 269:515-520, 1994.

6. Zeig M H, Campbell G. Receiver operating characteristics (ROC) plots: a fundamental evaluation tool in clinical medicine. Clin Chem 39: 561-577, 1993.

We claim:

1. A diagnostic kit for determining the concentration of 13(S)-HODE in a biological sample of a subject comprising:
(a) enzyme conjugated13(S)-HODE;
(b) enzyme-conjugated 13(S)-HODE substrate; and
(c) anti-13(S)-HODE specific antibody.

2. The diagnostic kit of claim 1 wherein the enzyme conjugated 13(S)-HODE is selected from the group consisting of 13(S)-HODE-horseradish peroxidase and 13(S)-HODE alkaline phosphatase.

3. The diagnostic kit of claim 1 wherein the enzyme-conjugated 13(S)-HODE substrate is a capable of undergoing a calorimetric change.

4. The diagnostic kit of claim 1 wherein the enzyme-conjugated 13(S)-HODE substrate is a capable of emitting a fluorescent signal.

5. A diagnostic kit for determining the concentration of 13(S)-HODE in a biological sample of a subject comprising:
(a) fluorochrome conjugated13(S)-HODE; and
(b) anti-13(S)-HODE specific antibody.

6. A diagnostic kit for determining the concentration of 13(S)-HODE in a biological sample of a subject comprising:
(a) radioisotope conjugated 13(S)-HODE; and
(b) anti-13(S)-HODE specific antibody.

7. The diagnostic kit of claim 1, 5 or 6 wherein the biological sample is selected from the group consisting of tissue fluid, blood, blood plasma, blood serum or urine.

8. The diagnostic kit of claim 1, 5 or 6 further comprising a standard.

9. The diagnostic kit of claim 8 wherein the standard comprises a known quantity of 13(S)-HODE.

10. The diagnostic kit of claim 1, 5 or 6 for use in assessing the risk or burden of the subject to cardiovascular disease.

11. The diagnostic kit of claim 1, 5 or 6 for use in assessing the thrombotic risk or burden of disease of the subject.

12. A diagnostic method to assess risk or burden of cardiovascular disease in a subject, wherein the concentration of 13(S)-HODE in a biological sample is determined, the method comprising the steps of:
(a) extracting 13(S)-HODE from a biological sample;
(b) incubating extracted 13(S)-HODE with enzyme-conjugated 13(S)-HODE and anti-13(S)-HODE specific antibody bound to an insoluble support;
(c) incubating the mixture resultant from step (b) with enzyme-conjugated 13(S)-HODE substrate;
(d) determining by optical density measurements the concentration of 13(S)-HODE that was present in the biological sample based on the color signal produced; and
(e) comparing the concentration of 13(S)-HODE that was present in the biological sample to the concentration of 13(S)-HODE in biological samples from healthy subjects.

13. A diagnostic method to assess risk or burden of cardiovascular disease in a subject, wherein the concentration of 13(S)-HODE in a biological sample is determined, the method comprising the steps of:
(a) extracting 13(S)-HODE from a biological sample;
(b) incubating extracted 13(S)-HODE with enzyme-conjugated 13(S)-HODE and anti-13(S)-HODE specific antibody bound to an insoluble support;
(c) incubating the mixture resultant from step (b) with enzyme-conjugated 13(S)-HODE substrate;
(d) determining by fluorescence the concentration of 13(S)-HODE that was present in the biological sample based on the fluorescent signal produced; and
(e) comparing the concentration of 13(S)-HODE that was present in the biological sample to the concentration of 13(S)-HODE in biological samples from healthy subjects.

14. A diagnostic method to asses risk or burden of cardiovascular disease in a subject, wherein the concentration of 13(S)-HODE in a biological sample is determined, the method comprising the steps of:
(a) extracting 13(S)-HODE from a biological sample;
(b) incubating extracted 13(S)-HODE with fluorochrome conjugated 13(S)-HODE and anti-13(S)-HODE specific antibody bound to an insoluble support;
(c) determining by fluorescence the concentration of 13(S)-HODE that was present in the biological sample based on the fluorescent signal produced; and
(d) comparing the concentration of 13(S)-HODE that was present in the biological sample to the concentration of 13(S)-HODE in biological samples from healthy subjects.

15. A diagnostic method to assess risk or burden of cardiovascular disease in a subject, wherein the concentration of 13(S)-HODE in a biological sample is determined, the method comprising the steps of:
(a) extracting 13(S)-HODE from a biological sample;
(b) incubating extracted 13(S)-HODE with radioisotope conjugated 13(S)-HODE and anti-13(S)-HODE specific antibody bound to an insoluble support;
(c) determining by radiation spectrometry the concentration of 13(S)-HODE that was present in the biological sample based on the radiation produced; and
(d) comparing the concentration of 13(S)-HODE that was present in the biological sample to the concentration of 13(S)-HODE in biological samples from healthy subjects.

16. A diagnostic method to assess thrombotic risk or burden of disease in a subject, wherein the concentration of 13(S)-HODE in a biological sample is determined, the method comprising the steps of:
(a) extracting 13(S)-HODE from a biological sample;
(b) incubating extracted 13(S)-HODE with enzyme-conjugated 13(S)-HODE and anti-13(S)-HODE specific antibody bound to an insoluble support;
(c) incubating the mixture resultant from step (b) with enzyme-conjugated 13(S)-HODE substrate;
(d) determining by optical density measurements the concentration of 13(S)-HODE that was present in the biological sample based on the color signal produced; and
(e) comparing the concentration of 13(S)-HODE that was present in the biological sample to the concentration of 13(S)-HODE in biological samples from healthy subjects.

17. A diagnostic method to assess thrombotic risk or burden of disease in a subject, wherein the concentration of 13(S)-HODE in a biological sample is determined, the method comprising the steps of:
(a) extracting 13(S)-HODE from a biological sample;
(b) incubating extracted 13(S)-HODE with enzyme-conjugated 13(S)-HODE and anti-13(S)-HODE specific antibody bound to an insoluble support;
(c) incubating the mixture resultant from step (b) with enzyme-conjugated 13(S)-HODE substrate;
(d) determining by fluorescence the concentration of 13(S)-HODE that was present in the biological sample based on the fluorescent signal produced; and
(e) comparing the concentration of 13(S)-HODE that was present in the biological sample to the concentration of 13(S)-HODE in biological samples from healthy subjects.

18. A diagnostic method to asses thrombotic risk or burden of disease in a subject, wherein the concentration of 13(S)-HODE in a biological sample is determined, the method comprising the steps of:
(a) extracting 13(S)-HODE from a biological sample;
(b) incubating extracted 13(S)-HODE with fluorochrome conjugated 13(S)-HODE and anti-13(S)-HODE specific antibody bound to an insoluble support;
(c) determining by fluorescence the concentration of 13(S)-HODE that was present in the biological sample based on the fluorescent signal produced; and
(d) comparing the concentration of 13(S)-HODE that was present in the biological sample to the concentration of 13(S)-HODE in biological samples from healthy subjects.

19. A diagnostic method to assess thrombotic risk or disease in a subject, wherein the concentration of 13(S)-HODE in a biological sample is determined, the method comprising the steps of:
(a) extracting 13(S)-HODE from a biological sample;
(b) incubating extracted 13(S)-HODE with radioisotope conjugated 13(S)-HODE and anti-13(S)-HODE specific antibody bound to an insoluble support;
(c) determining by radiation spectrometry the concentration of 13(S)-HODE that was present in the biological sample based on the radiation produced; and
(d) comparing the concentration of 13(S)-HODE that was present in the biological sample to the concentration of 13(S)-HODE in biological samples from healthy subjects.

20. A diagnostic kit for determining the concentration of 13(R)-HODE in a biological sample of a subject comprising:
(a) enzyme conjugated 13(R)-HODE;
(b) enzyme-conjugated 13(R)-HODE substrate; and
(c) anti-13(R)-HODE specific antibody.

21. The diagnostic kit of claim 20 wherein the enzyme conjugated 13(R)-HODE is selected from the group consisting of 13(R)-HODE-horseradish peroxidase and 13(R)-HODE alkaline phosphatase.

22. The diagnostic kit of claim 20 wherein the enzyme-conjugated 13(R)-HODE substrate is a capable of undergoing a colorimetric change.

23. The diagnostic kit of claim 20 wherein the enzyme-conjugated 13(R)-HODE substrate is a capable of emitting a fluorescent signal.

24. A diagnostic kit for determining the concentration of 13(R)-HODE in a biological sample of a subject comprising:
(a) fluorochrome conjugated 13(R)-HODE; and
(b) anti-13(R)-HODE specific antibody.

25. A diagnostic kit for determining the concentration of 13(R)-HODE in a biological sample of a subject comprising:
(a) radioisotope conjugated 13(R)-HODE; and
(b) anti-13(R)-HODE specific antibody.

26. The diagnostic kit of claim 1, 5 or 6 wherein the biological sample is selected from the group consisting of tissue fluid, blood, blood plasma, blood serum or urine.

27. The diagnostic kit of claim 1, 5 or 6 further comprising a standard.

28. The diagnostic kit of claim 8 wherein the standard comprises a known quantity of 13(R)-HODE.

29. The diagnostic kit of claim 1, 5 or 6 for use in assessing the risk or burden of the subject to cardiovascular disease.

30. The diagnostic kit of claim 1, 5 or 6 for use in assessing the thrombotic risk or burden of disease of the subject.

31. A diagnostic method to assess risk or burden of cardiovascular disease in a subject, wherein the concentration of 13(R)-HODE in a biological sample is determined, the method comprising the steps of:
(a) extracting 13(R)-HODE from a biological sample;
(b) incubating extracted 13(R)-HODE with enzyme-conjugated 13(R)-HODE and anti-13(R)-HODE specific antibody bound to an insoluble support;
(c) incubating the mixture resultant from step (b) with enzyme-conjugated 13(R)-HODE substrate;
(d) determining by optical density measurements the concentration of 13(R)-HODE that was present in the biological sample based on the color signal produced; and
(d) comparing the concentration of 13(R)-HODE that was present in the biological sample to the concentration of 13(R)-HODE in biological samples from healthy subjects.

32. A diagnostic method to assess risk or burden of cardiovascular disease in a subject, wherein the concentration of 13(R)-HODE in a biological sample is determined, the method comprising the steps of:
(a) extracting 13(R)-HODE from a biological sample;
(b) incubating extracted 13(R)-HODE with enzyme-conjugated 13(R)-HODE and anti-13(R)-HODE specific antibody bound to an insoluble support;
(c) incubating the mixture resultant from step (b) with enzyme-conjugated 13(R)-HODE substrate;
(d) determining by fluorescence the concentration of 13(R)-HODE that was present in the biological sample based on the fluorescent signal produced; and
(e) comparing the concentration of 13(R)-HODE that was present in the biological sample to the concentration of 13(R)-HODE in biological samples from healthy subjects.

33. A diagnostic method to asses risk or burden of cardiovascular disease in a subject, wherein the concentration of 13(R)-HODE in a biological sample is determined, the method comprising the steps of:
(a) extracting 13(R)-HODE from a biological sample;
(b) incubating extracted 13(R)-HODE with fluorochrome conjugated 13(R)-HODE and anti-13(R)-HODE specific antibody bound to an insoluble support;
(c) determining by fluorescence the concentration of 13(R)-HODE that was present in the biological sample based on the fluorescent signal produced; and
(d) comparing the concentration of 13(R)-HODE that was present in the biological sample to the concentration of 13(R)-HODE in biological samples from healthy subjects.

34. A diagnostic method to assess risk or burden of cardiovascular disease in a subject, wherein the concentration of 13(R)-HODE in a biological sample is determined, the method comprising the steps of:
(a) extracting 13(R)-HODE from a biological sample;
(b) incubating extracted 13(R)-HODE with radioisotope conjugated 13(R)-HODE and anti-13(R)-HODE specific antibody bound to an insoluble support;
(c) determining by radiation spectrometry the concentration of 13(R)-HODE that was present in the biological sample,based on the radiation produced; and
(d) comparing the concentration of 13(R)-HODE that was present in the biological sample to the concentration of 13(R)-HODE in biological samples from healthy subjects.

35. A diagnostic method to assess thrombotic risk or burden of disease in a subject, wherein the concentration of 13(R)-HODE in a biological sample is determined, the method comprising the steps of:
(a) extracting 13(R)-HODE from a biological sample;
(b) incubating extracted 13(R)-HODE with enzyme-conjugated 13(R)-HODE and anti-13(R)-HODE specific antibody bound to an insoluble support;
(c) incubating the mixture resultant from step (b) with enzyme-conjugated 13(R)-HODE substrate;
(d) determining by optical density measurements the concentration of 13(R)-HODE that was present in the biological sample based on the color signal produced; and
(e) comparing the concentration of 13(R)-HODE that was present in the biological sample to the concentration of 13(R)-HODE in biological samples from healthy subjects.

36. A diagnostic method to assess thrombotic risk or burden of disease in a subject, wherein the concentration of 13(R)-HODE in a biological sample is determined, the method comprising the steps of:
(a) extracting 13(R)-HODE from a biological sample;
(b) incubating extracted 13(R)-HODE with enzyme-conjugated 13(R)-HODE and anti-13(R)-HODE specific antibody bound to an insoluble support;
(c) incubating the mixture resultant from step (b) with enzyme-conjugated 13(R)-HODE substrate;
(d) determining by fluorescence the concentration of 13(R)-HODE that was present in the biological sample based on the fluorescent signal produced; and
(e) comparing the concentration of 13(R)-HODE that was present in the biological sample to the concentration of 13(R)-HODE in biological samples from healthy subjects.

37. A diagnostic method to asses thrombotic risk or burden of disease in a subject, wherein the concentration of 13(R)-HODE in a biological sample is determined, the method comprising the steps of:
(a) extracting 13(R)-HODE from a biological sample;
(b) incubating extracted 13(R)-HODE with fluorochrome conjugated 13(R)-HODE and anti-13(R)-HODE specific antibody bound to an insoluble support;
(c) determining by fluorescence the concentration of 13(R)-HODE that was present in the biological sample based on the fluorescent signal produced; and
(d) comparing the concentration of 13(R)-HODE that was present in the biological sample to the concentration of 13(R)-HODE in biological samples from healthy subjects.

38. A diagnostic method to assess thrombotic risk or burden of disease in a subject, wherein the concentration of 13(R)-HODE in a biological sample is determined, the method comprising the steps of:
(a) extracting 13(R)-HODE from a biological sample;
(b) incubating extracted 13(R)-HODE with radioisotope conjugated 13(R)-HODE and anti-13(R)-HODE specific antibody bound to an insoluble support;
(c) determining by radiation spectrometry the concentration of 13(R)-HODE that was present in the biological sample based on the radiation produced; and
(d) comparing the concentration of 13(R)-HODE that was present in the biological sample to the concentration of 13(R)-HODE in biological samples from healthy subjects.
